(19) United States
(12) Patent Application Publication
(10) Pub. No.: US 2015/0038726 A1
(43) Pub. Date: Feb. 5, 2015

(54) PROCESS FOR THE PREPARATION OF CARBAPENEM ANTIBIOTIC

(71) Applicant: HOSPIRA, INC., Lake Forest, IL (US)

(72) Inventors: Senthilkumar Udayampalayam Palanisamy, Sozhanganallur (IN); Sureshkumar Kanagaraj, Sozhanganallur (IN); Nagesh Babu Kommoju, Sozhanganallur (IN); Syril Sudhan Henry, Sozhanganallur (IN); Pravin Kamaraj Ponraj, Sozhanganallur (IN); Suresh Thangaiyan, Sozhanganallur (IN)

(21) Appl. No.: 14/390,704

(22) PCT Filed: Apr. 4, 2013
(86) PCT No.: PCT/IN2013/000229

§ 371 Date: Oct. 3, 2014

(30) Foreign Application Priority Data
Apr. 4, 2012 (IN) 1371/CHE/2012
Dec. 7, 2012 (IN) 5111/CHE/2012

(51) Int. Cl. C07D 487/04

(52) U.S. Cl. CPC C07D 487/04; C07B 2200/13
USPC 548/467

(57) ABSTRACT

The present invention provides an improved process for the preparation Ertapenem monosodium of formula (I) having purity greater than 98.5% and having pharmaceutically acceptable level of palladium and residual solvent. (I)

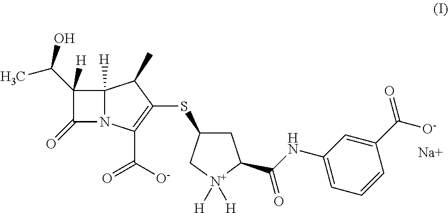 [SMILES: C[C@@H](O)[C@H]1C(=O)N2C(C(=O)[O-])=C(S[C@@H]3C[NH2+][C@H](C(=O)Nc4cccc(C(=O)[O-])c4)C3)[C@H](C)[C@H]12 ; [Na+]]

### FIELD OF THE INVENTION

The present invention relates to an improved process for the preparation of carbapenem antibiotic; more particularly relates to the preparation of Ertapenem monosodium salt of formula (I) having purity greater than 98.5% and having pharmaceutically acceptable level of residual solvent and palladium content.

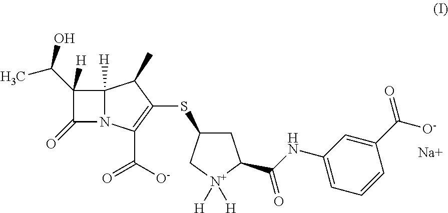 [SMILES: C[C@@H](O)[C@H]1C(=O)N2C(C(=O)[O-])=C(S[C@@H]3C[NH2+][C@H](C(=O)Nc4cccc(C(=O)[O-])c4)C3)[C@H](C)[C@H]12 ; [Na+]]

### BACKGROUND OF THE INVENTION

Ertapenem is a β-methylcarbapenem marketed by Merck as Invanz®. The chemical name of Ertapenem sodium is [4R-[3(3S*,5S*),4α,5β,6β(R*)]]-3-[[5-[[(3-carboxyphenyl)amino]carbonyl]-3-pyrrolidinyl]thio]-6-(1-hydroxyethyl)-4-methyl-7-oxo-1-azabicyclo[3.2.0]hept-2-ene-2-carboxylic acid monosodium salt.

Ertapenem monosodium of formula (I) is a 1β-methylcarbapenem antibiotic, and used as an antibiotic agent in the treatment of moderate to severe complicated foot infection due to indicated pathogens in diabetic patients without osteomyelitis. Ertapenem is also useful in the treatment of pneumonia, urinary tract infections, intra-abdominal, gynecological, skin, and soft tissue infections, meningitis, septicemia and febrile Neutrogena.

In view of the importance of the Ertapenem monosodium, several synthetic procedures have been reported.

The US patents namely U.S. Pat. No. 5,478,820 and U.S. Pat. No. 5,856,321 disclose various processes for preparing Ertapenem and its sodium salt. Example 12 of U.S. Pat. No. 5,478,820 discloses a process in which the Ertapenem was isolated using column purification followed by freeze-drying technique. According to Example-4 of this patent disodium salt of Ertapenem was prepared by dissolving crude product in water using NaHCO₃, followed by purification using column chromatography and subsequent lyophilization.

U.S. Pat. No. 6,504,027 provides a process for preparing Ertapenem in crystalline form which comprises deprotecting and extracting a polar organic solution containing a crude mono-protected Ertapenem of formula

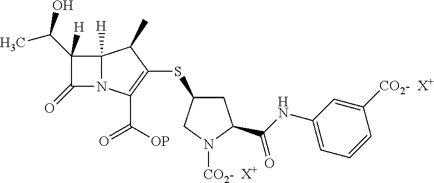

wherein P represents protecting group and X represents charge balancing group like sodium
with C₄₋₁₀ alcohol in the presence of ion-pairing reagent followed by adjusting the pH of the aqueous layer to 5.5 and crystallizing using methanol and 1-propanol to produce a crystalline compound; this patent process involves operations like multiple extractions which is cumbersome in plant and said operation affects the overall yield.

U.S. Pat. No. 7,145,002 provides a process for producing Ertapenem or its sodium salt and/or its solvate in crystalline form. This patent states (refer para 3, lines 31-41) that contact of Ertapenem sodium with water and alcoholic solvents results in the formation of crystalline solvates. The processes reported in examples-1 & 2 provide crystalline Ertapenem monosodium which is isolated from a mixture of methanol, 1-propanol and water followed by washing with aqueous isopropyl alcohol which results in the formation of crystalline solvate of Ertapenem sodium. Applicant found the Ertapenem monosodium obtained according to this process contain higher amount of residual solvent and palladium content.

U.S. Pat. No. 7,022,841 provide a process for reducing the levels of organic solvents in Ertapenem to pharmaceutically acceptable levels. This patent discloses (Refer para 1, lines 52-60) that Ertapenem sodium obtained from water/alcohol mixture according to U.S. Pat. No. 7,145,002 becomes amorphous when water content of the solid is reduced and further the organic solvent present in the solid is not readily removed. In view of this drawback, this patent provides a process wherein the water content of Ertapenem sodium is maintained between 13-25% during the washing and drying process. This patent further discloses that (Refer para 9, lines 6-14) the washing of Ertapenem sodium can be carried out using anhydrous solvents which results in the formation of amorphous solid, which is then dried using hydrated nitrogen by increasing the water content of the solid. Due to the hygroscopic and unstable nature of Ertapenem sodium when in contact with water, the above processes result in more degradation of Ertapenem. The patent further discloses in example 5 that the degradation of Ertapenem sodium is more when it takes more time for drying. Further this patent requires repetitive washing and control of moisture content to get the desired results.

For isolation of Ertapenem sodium from the reaction mass, all the above discussed prior art patents utilize methanol and 1-propanol as crystallization solvent. The filtration of Ertapenem sodium formed by using these solvents or their mixture takes longer time duration and subsequent drying for the removal of residual solvent also takes several hours due to occlusion of solvent into Ertapenem sodium. During these operations the Ertapenem sodium degrades and results in the formation of many impurities such as several dimers, methanolysis impurity etc., and hence the reported processes is not suitable to manufacture Ertapenem sodium on commercial scale with purity greater than 98.5% and with pharmaceutically acceptable level of residual solvent content.

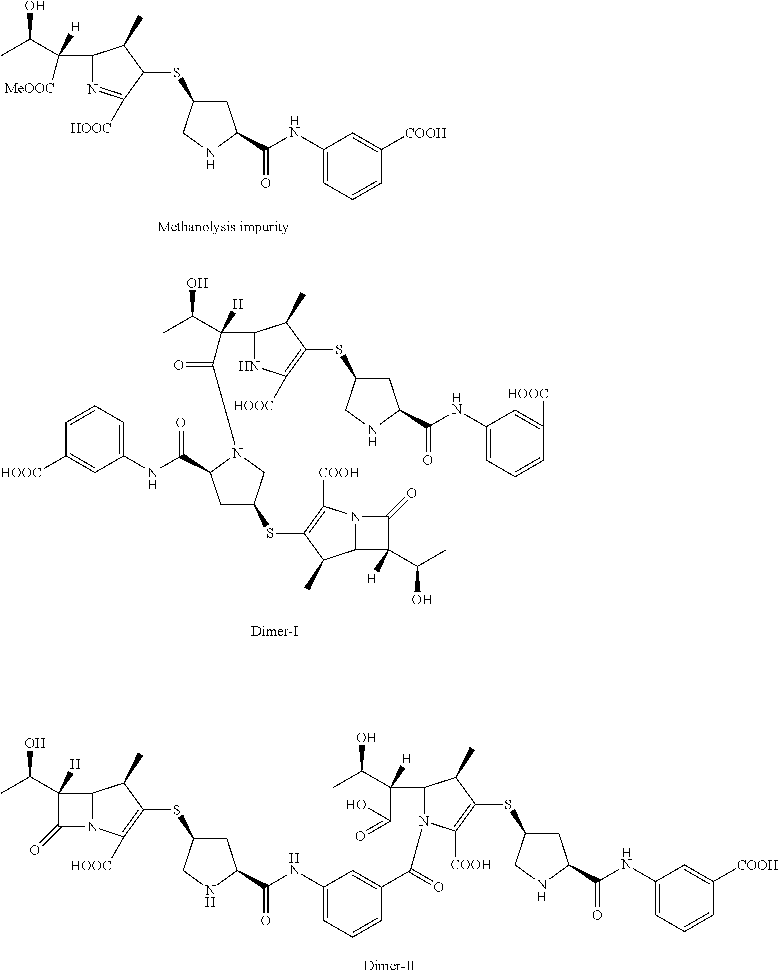 [SMILES: COC(=O)[C@@H](C1N=C(C(=O)O)C(S[C@@H]2CN[C@H](C(=O)Nc3cccc(C(=O)O)c3)C2)[C@@H]1C)[C@@H](C)O ; C[C@H]1C(S[C@@H]2CN[C@H](C(=O)Nc3cccc(C(=O)O)c3)C2)=C(C(=O)O)NC1[C@H](C(=O)N1C[C@@H](SC2=C(C(=O)O)N3C(=O)[C@H]([C@@H](C)O)C3[C@H]2C)C[C@H]1C(=O)Nc1cccc(C(=O)O)c1)[C@@H](C)O ; C[C@H]1C(S[C@@H]2CN[C@H](C(=O)Nc3cccc(C(=O)O)c3)C2)=C(C(=O)O)N(C(=O)c2cccc(NC(=O)[C@@H]3C[C@H](SC4=C(C(=O)O)N5C(=O)[C@H]([C@@H](C)O)C5[C@H]4C)CN3)c2)C1[C@H](C(=O)O)[C@@H](C)O]

Further the applicant found that Ertapenem monosodium isolated by following the process reported in prior art was having palladium content above the pharmaceutically acceptable level. Hence the process reported in prior art is not suitable on manufacturing scale where maintaining stringent technological condition is cumbersome and involves higher operating cost.

Thus all the reported processes suffer in terms of one or more of the following facts:
Filtration time of Ertapenem sodium takes several hours.
Drying time takes several hours due to occlusion of solvent and nature of the solid.
Stringent technological condition is required for maintenance of moisture content during washing & drying operation.
Palladium content is found to be higher (greater than 25 ppm) which is not acceptable for pharmaceutical products.
The isolated Ertapenem sodium is having higher amount of residual solvents.
The purity is reduced over to several hours of filtration & drying.

With our continued research for developing a process for the preparation of Ertapenem monosodium of formula (I) to overcome the above mentioned drawbacks, we surprisingly found that when esters of organic acid were used as solvents in place of 1-propanol, the solid obtained was easily filterable with less cycle time. Further the washing with hydrocarbon solvents containing 0-75% alcoholic solvent followed by drying results in Ertapenem having residual solvent, content well below the pharmaceutically acceptable levels. The use of thiourea, thiosemicarbazide or their N-substituted derivatives in the presence of organic solvents during isolation brings down the palladium content to pharmaceutically acceptable level.

### Objectives of the Invention

The main objective of the present invention is to provide a simple, robust, commercially viable, and industrially scalable process for the preparation of Ertapenem monosodium of formula (I), which avoids techniques like column chromatography, freeze-drying, special operating conditions like maintaining water content during filtration & drying.

One more objective is to provide process for the preparation of life-saving antibiotic, namely Ertapenem sodium, with high quality.

Another objective of the present invention is to provide Ertapenem monosodium of formula (I), having residual solvent content well within the pharmaceutically acceptable level.

Still another objective of the present invention is to prepare Ertapenem monosodium of formula (I) that avoids the longer time consuming process disclosed in prior arts thereby increasing the productivity, apart from high quality.

Yet another objective of the present invention is to provide Ertapenem sodium having pharmaceutically acceptable level of palladium by using thiourea, thiosemicarbazide or their N-substituted derivatives.

One another objective of the present invention is to provide a novel crystalline form of Ertapenem monosodium characterized by having X-ray powder diffraction pattern as given in FIG. 1 and having peaks at 4.3, 5.2, 7.4, 7.9, 8.6, 9.3 and 10.8 (±0.20) in 2θ.

### SUMMARY OF THE INVENTION

Accordingly, the present invention provides an improved process for the preparation of Ertapenem monosodium of formula (I) having purity greater than 98.5%;

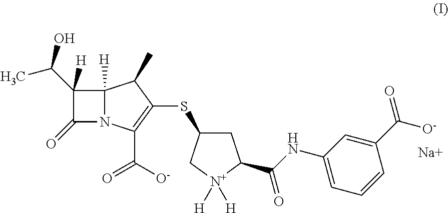 [SMILES: C[C@@H](O)[C@H]1C(=O)N2C(C(=O)[O-])=C(S[C@@H]3C[NH2+][C@H](C(=O)Nc4cccc(C(=O)[O-])c4)C3)[C@H](C)[C@H]12 ; [Na+]]

the said process comprises the steps of:
(a) obtaining an aqueous solution containing Ertapenem monosodium of formula (I);
(b) combining the step (a) mass with alcoholic solvent;
(c) mixing ester of an organic acid with the step (b) mass; and
(d) filtering the solid followed by washing the solid with hydrocarbon solvent containing 0-75% of alcoholic solvent.

An another aspect of the present invention is to provide a crystalline form of Ertapenem monosodium of formula (I) characterized by having X-ray diffraction pattern in FIG. 1 and having peaks at 4.3, 5.2, 7.4, 7.9, 8.6, 9.3 and 10.8 (±0.20) in 2θ.

Still another aspect is of the present invention is to provide a process for reducing the palladium content in penem/penam antibiotic comprising treating the aqueous solution of penem/penam antibiotic with thiourea, thiosemicarbazide or their N-substituted derivatives in the presence of an organic solvent followed by isolation of the penem/penam antibiotic by conventional methods.

### BRIEF DESCRIPTION OF THE FIGURE

FIG. 1: X-ray diffractogram of Ertapenem monosodium of the present invention The PXRD is measured using Diffractometer of following features:

| | |
|---|---|
| Make | BRUKER AXS |
| Model | D8 ADVANCE |
| Data handling system | EVA 12.0.0.0. |
| ANODE | COPPER |
| RADIATION | COPPER K alpha |
| WAVELENGTH | 1.5406 A° |
| CURRENT &VOLTAGE | 30 mA & 40 kV |

### DESCRIPTION OF THE INVENTION

The aqueous solution containing Ertapenem monosodium can be obtained directly from the reaction mass or by dissolving Ertapenem monosodium in water or by dissolving Ertapenem disodium or its carbamate in water followed by adjusting the pH to 5-6 to get a solution of Ertapenem monosodium. The monosodium and disodium of Ertapenem can be characterized by determining the sodium content. The sodium content of Ertapenem monosodium and disodium be in the range of about 4.5-4.8 and 8.7-9.0 respectively. The resultant aqueous solution containing Ertapenem monosodium can be optionally washed with organic solvents like dichloromethane, n-butyl acetate, ethyl acetate, toluene, hexane, 1,2-dibromoethane, tetrahydrofuran, 2-methyltetrahydrofuran or mixtures thereof to remove reaction by-products and/or impurities. If required carbon dioxide gas can be purged to the aqueous solution. The aqueous solution containing Ertapenem monosodium can be subjected to degassing technique to remove any dissolved solvent, if necessary by using vacuum or by purging nitrogen. For Ertapenem monosodium, the pH of aqueous layer is adjusted to 5.0 to 6.0 using acids like acetic acid, formic acid or hydrochloric acid.

In one more embodiment of the present invention, the alcoholic solvent used in step (b) is selected from methanol, ethanol or isopropyl alcohol, preferably methanol. The alcoholic solvent is added in such way to get a homogeneous solution before the addition of ester of an organic acid.

In yet another embodiment of the present invention the ester of an organic acid used in step (c) to crystallize the Ertapenem monosodium of formula (I) is selected from ethyl acetate, methyl acetate, n-propyl acetate, isopropyl acetate, n-butyl acetate and the like, preferably ethyl acetate. Applicant surprisingly found that the use of ester of an organic acid like ethyl acetate for the crystallization helps to reduce the filtration time and thereby the purity of Ertapenem monosodium obtained is higher than 98.5% by HPLC. Accordingly the use of ethyl acetate as crystallization solvent constitutes one of the novelty rendering feature of the present invention.

In one more embodiment of the present invention, the washing solvent used in step (d) is selected from hydrocarbon solvent containing 0-75% of alcoholic solvent. The hydrocarbon solvent is selected from linear alkanes, cyclic alkanes or aromatic hydrocarbons, preferably cyclohexane, cyclopentane, decalin, n-hexane, n-heptane or toluene, more preferably cyclohexane. The said hydrocarbon solvent may contain 0 to 75% of alcoholic solvent such as methanol, ethanol, isopropanol, methoxy ethanol preferably ethanol. Applicant observed that the use of washing and drying processes reported in prior arts failed to remove the residual solvent(s) in Ertapenem monosodium or the said techniques are not industrially viable or it require repetitive washing. Surprisingly the use of hydrocarbon solvent containing 0 to 75% of alcoholic solvent helps in the removal of residual solvent in Ertapenem monosodium to pharmaceutically acceptable level. The said washing and subsequent drying process do not require maintenance of water content. With this process, the drying time is reduced drastically when compared to the prior art processes. Accordingly the present invention provides an improved process for reducing the residual solvent in Ertapenem monosodium which comprises washing the Ertapenem monosodium containing residual solvents selected from the group consisting of ethyl acetate, methanol, tetrahydrofuran, 2-methyltetrahydrofuran, N,N-dimethylformamide, N,N-dimethylacetamide, 1-propanol, isopropanol, dichloromethane, methyl acetate, propyl acetate, butyl acetate, acetonitrile, toluene, hexane, 1,2-dibromoethane or mixture thereof with hydrocarbon solvent containing 0 to 75% of alcoholic solvent. The term “pharmaceutically acceptable level/limit” means the level of parameters like residual organic solvent/palladium content which is less than the amount recommended for pharmaceutical products, as set forth for example in ICH guidelines and U.S. Pharmacopoeia or other such regulatory bodies.

In another embodiment of the present invention, the processes provided in the present invention can be used to prepare sterile Ertapenem monosodium. This can be carried out by micron filtration of the aqueous solution containing Ertapenem monosodium before the addition of anti-solvent in sterile area. Micron filtration is a membrane filtration process which removes contaminants like bacteria, particles and sediment from a liquid by passage through a microporous membrane.

In one another embodiment of the present invention, the process provided in the present invention can be used to purify crude Ertapenem. This can be achieved by dissolving the crude Ertapenem sodium in water followed by adding the aqueous solution in to alcoholic solvent and crystallization by adding ester of organic acid. Further the washing can be carried out using hydrocarbon solvent containing 0-75% alcoholic solvent.

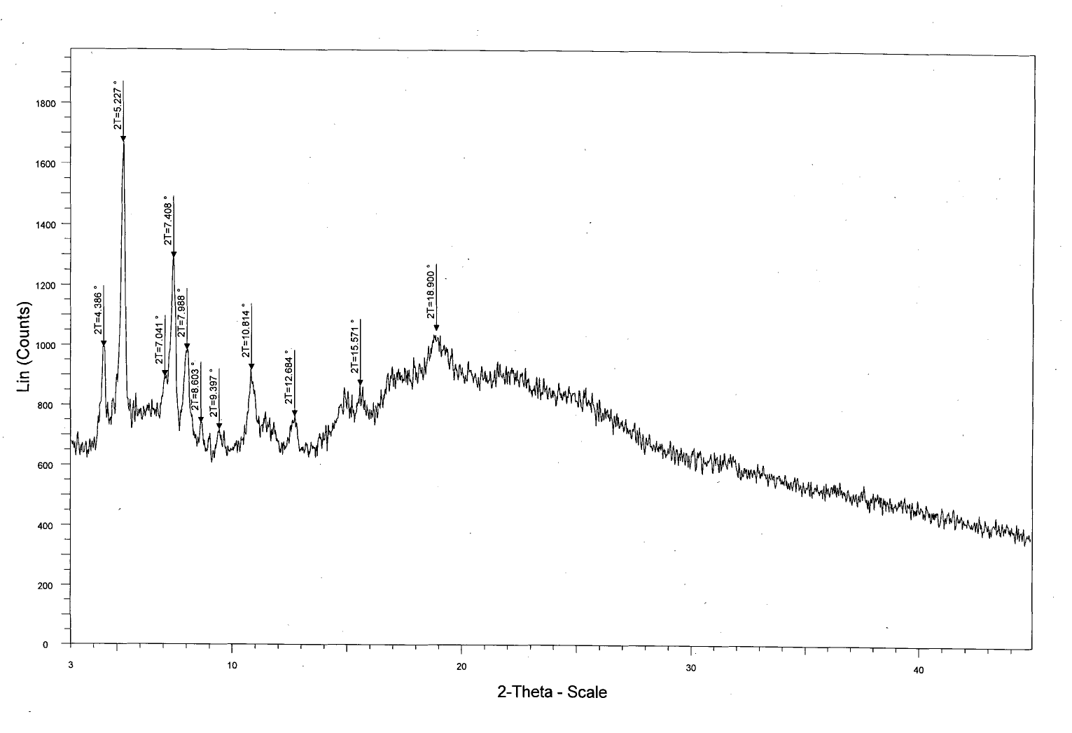

In yet another embodiment of the present invention the crystalline form of Ertapenem monosodium obtained by the present invention is characterized by X-ray diffraction pattern in FIG. 1 and having peaks at 4.3, 5.2, 7.4, 7.9, 8.6, 9.3 and 10.8 (±0.20) in 2θ.

Applicant found that the Ertapenem monosodium isolated according to the present invention or by the processes reported in prior art contains higher palladium content i.e., greater than 25 ppm, which is higher than the pharmaceutically acceptable limit. Being an injectable product, the palladium content in the final compound should be less than 7 ppm calculated on the basis of maximum daily dosage of Ertapenem. The conventional reported process such as use of EDTA and polymer supported resin for reduction of palladium fails to produce desired result. Thus applicant diligently worked and found that the contact of aqueous solution of Ertapenem sodium with thiourea, thiosemicarbazide or their N-substituted derivatives in the presence of organic solvents results in the reduction of palladium content and isolation of Ertapenem monosodium with pharmaceutically acceptable level of palladium while maintaining the purity greater than 98.5%. The beta-lactam moiety of carbapenems is known to be prone to attack by wide range of nucleophiles, including weaker nucleophiles owing to strain in the four-member ring in bicyclic system. Despite that there is a possibility of degradation of Ertapenem monosodium when using nucleophilic reagents like thiourea or thiosemicarbazide or their N-substituted derivatives with the formation of ring-open impurity, applicant successfully found a suitable condition wherein the Ertapenem monosodium obtained with purity greater than 98.5% and palladium content lower than 7 ppm.

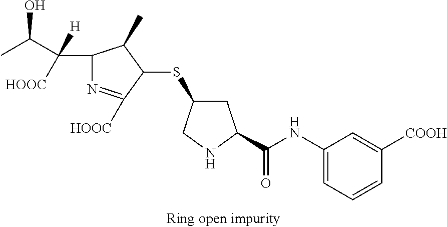 [SMILES: C[C@H]1C(S[C@@H]2CN[C@H](C(=O)Nc3cccc(C(=O)O)c3)C2)C(C(=O)O)=NC1[C@H](C(=O)O)[C@@H](C)O]

The aqueous solution of carbapenem like Ertapenem having higher amount of palladium is treated with thiourea or thiosemicarbazide or their N-substituted derivatives in the presence of organic solvent for a period of 5 minutes to 2 hours. The N-substituent includes but not limited to N-alkyl, N-aryl, N-alkenyl, N-acetyl, N-alkynyl, N-sulfonyl, N-carbamoyl or N-cyclic, N-amino, N-aralkyl, N-cyano etc. The amount of the reagents used may vary from 0.01% to 20%. Thus the present invention further provides an improved process for the preparation of Ertapenem monosodium of formula (I) with reduced content of palladium which comprises the steps of:
i) obtaining aqueous solution containing Ertapenem monosodium of formula (I);
ii) contacting the solution with thiourea or thiosemicarbazide or their N-substituted derivatives in the presence of organic solvent;
iii) combining step (ii) mass with alcoholic solvent;
iv) mixing anti-solvent with the step (iii) mass;
v) isolating the Ertapenem monosodium salt.

In another embodiment of the present invention, the organic solvent used in step (ii) is selected from tetrahydrofuran, 2-methyltetrahydrofuran, dichloromethane, butyl acetate, ethyl acetate, isopropyl acetate, acetonitrile, toluene, hexane, 1,2-dibromoethane or mixtures thereof, preferably tetrahydrofuran and 2-methyltetrahydrofuran more preferably 2-methyltetrahydrofuran.

In one more embodiment of the present invention, the alcoholic solvent used in step (iii) is selected from methanol, ethanol or isopropyl alcohol or mixtures thereof; preferably methanol.

In yet another embodiment of the present invention, the anti-solvent used in step (iv) is selected from ethanol, isopropyl alcohol, 1-propanol, ethyl acetate, methyl acetate, n-propyl acetate, isopropyl acetate, n-butyl acetate or the mixtures thereof.

Applicant found that the present invention of use of thiourea derivative is also useful in reducing the palladium content in the preparation of penem/penam antibiotic where the final step involves the use of palladium; the said process comprises treating the aqueous solution of penem/penam antibiotic with thiourea or thiosemicarbazide or their N-substituted derivatives in the presence of organic solvent followed by isolating the penem/penam antibiotic from aqueous layer by conventional methods. The penem/penam antibiotic is selected from Ertapenem or it monosodium salt, Meropenem or its hydrate, Imipenem or its hydrate, Biapenem or its hydrate and Doripenem or its hydrate, Tebipenem, Faropenem, Tazobactam etc. The use of thiourea derivatives in the isolation of above said penem/penam molecules helps to reduce the palladium content significantly. All these molecules contain beta-lactam ring and hence susceptible to degradation in the presence of nucleophile like thiourea. Despite the above fact, applicant found that under suitable reaction condition, the use of thiourea or its derivatives helps to reduce palladium content in above said penem/penam antibiotics by avoiding the degradation.

In yet another embodiment of the present invention the aqueous solution of penem/penam antibiotics having higher amount of palladium content are obtained by deprotecting the protecting groups in precursor compounds using palladium or its complex and filtering the palladium or its complex.

In one more embodiment of the present invention the penem/penam antibiotic from aqueous solution, after reduction of palladium content, is isolated by conventional method like, but not limited to, adjusting the pH in the presence or absence of water miscible organic solvent, adding anti-solvent, extraction, cooling the aqueous solution in the presence or absence of water miscible organic solvent or combinations thereof.

The Ertapenem or its sodium salt can be prepared according the processes provided in prior arts or by following the scheme as shown below:

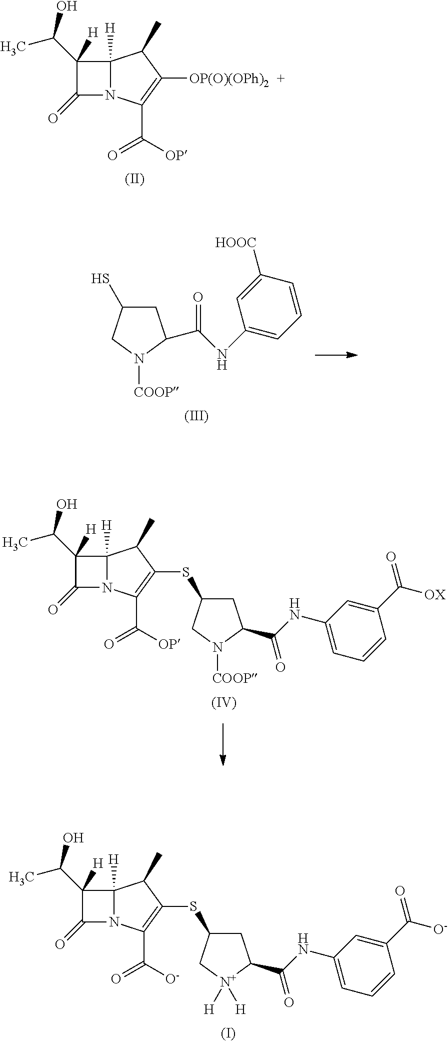

The present invention is illustrated with the following examples, which should not be construed to limit the scope of the invention.

### Example-I

### Preparation of Ertapenem Monosodium of Formula (I)

### Step-I

To a stirred solution of p-nitrobenzyl (4R,5S,6S)-3-(diphenyloxy)phosphoryloxy-6-[(1R)-1-hydroxyethyl]-4-methyl-7-oxo-1-azabicyclo[3,2,0]hept-2-ene-2-carboxylate (compound II) (100 g) and (2S,4S)-2-[[(3-carboxyphenyl)amino]carbonyl]-4-mercapto-1-(4-nitrobenzyl)pyrrolidinecarboxylate (compound III) (75 g) in N,N-dimethylformamide was added N,N-diisopropylethylamine at −30 to −40° C. and stirred. The reaction mass, after completion of the reaction, was quenched with a mixture of phosphate buffer solution-ethyl acetate and the pH was adjusted to 5-6 with phosphoric acid. The organic layer was separated, washed with water and subjected to carbon treatment. To the organic layer containing the compound of formula (IV) (wherein P′ and P″ refers to p-nitrobenzyl), a solution of sodium 2-ethylhexanoate (42 g in 500 mL methanol) was added and taken to next step as such. (If required the compound of formula (IV) is isolated either as sodium salt or as free acid by following the process reported in prior art and taken further)

### Step-II:

To the Step-I organic layer containing the compound of formula (IV) (wherein P′ and P″ refers to p-nitrobenzyl & X is Na), 3-(N-morpholino)propanesulfonic acid solution was added and subjected to hydrogenation using palladium on carbon at 8-10° C. with 9-10 kg hydrogen pressure. The reaction mass, after completion of reaction, was filtered to remove palladium on carbon. To the filtrate, thiourea (5 g) and tetrahydrofuran were added and stirred. The aqueous layer was separated and treated with carbon and neutral alumina at 10-15° C. while degassing and filtered. The filtrate was added to methanol at −20° C. and the pH was adjusted to 5-6 using aqueous acetic acid. To the mass, ethyl acetate was added and stirred. The solid obtained was filtered, washed with a mixture of cyclohexane:ethanol (200 ml) and dried under vacuum. Yield: 46 g; Purity by HPLC: 98.93%; Palladium content: 1.8 ppm by ICP MS

The HPLC purity of Ertapenem monosodium was checked using the following parameters
Column: Zorbax Eclipse plus C8, (50 mm×4.6 mm), 1.8μ).
Mobile phase: Ammoniam acetate buffer: Acetonitile: water
Detector: UV at 250 nm
Flow rate: 0.5 mL/min
Run time: 45 min.

### Example-II

### Preparation of Ertapenem Monosodium of Formula (I)

To the Step-I organic layer as provided in Example-I, 3-(N-morpholino)propanesulfonic acid solution was added and subjected to hydrogenation using palladium on carbon at 8-10° C. with 9-10 kg hydrogen pressure. The reaction mass, after completion of reaction, was filtered and the filtrate was treated with thiourea and 2-methyltetrahydrofuran and the layers separated. The aqueous layer was treated with carbon & neutral alumina at 10-15° C. and filtered. The filtrate was mixed with methanol at −20° C. and the pH was adjusted to 5-6 using aqueous acetic acid. To the mass, ethyl acetate was added and stirred. The solid obtained was filtered, washed with cyclohexane (200 ml) and dried under vacuum. Yield: 44 g; Purity by HPLC: 98.84%; Palladium content: 0.93 ppm by ICP MS

The term ICP MS method refers to the inductively coupled plasma mass spectrometry. The following parameter was used to determine the content of palladium.

The carbapenem was digested in a closed vessel system in presence of reagents Nitric acid, Hydrogen peroxide and Hydrochloric acid by using Microwave reaction system with microwave radiation power 1200 Watts. The digested sample was introduced into inductively coupled plasma mass spectrometer by help of Peltier cooled spray chamber. The sample aerosol is getting atomized then ionized in the argon plasma. The ionized Palladium was estimated by using Quadrupole mass detector. The sample was quantified against NIST traceable reference standards at mass number 105.

### Example-III

### Preparation of Ertapenem Monosodium of Formula (I)

To the Step-I organic layer as provided in Example-I, 3-(N-morpholino)propanesulfonic acid solution was added and hydrogenated at 9-10 kg pressure using palladium on carbon at 8-10° C. The reaction mass, after completion of reaction, was filtered and the filtrate was treated with thiourea and tetrahydrofuran and the layers separated. The aqueous layer was separated and treated with carbon, neutral alumina at 10-15° C. and filtered. The filtrate was mixed with methanol at −20° C. and the pH was adjusted to 5-6 using aqueous acetic acid. To the mass, ethyl acetate was added and stirred. The solid obtained was filtered, washed with a mixture of toluene: ethanol (200 ml) and dried under vacuum. Yield: 42 g; Purity by HPLC: 99.03%

### Example-IV

### Preparation of Ertapenem Monosodium of Formula (I)

To the Step-I organic layer as provided in Example-I, 3-(N-morpholino)propanesulfonic acid solution was added and hydrogenated using palladium on carbon at 8-10° C. The reaction mass, after completion of reaction was filtered and the filtrate was treated with thiosemicarbazide and tetrahydrofuran and the layers separated. The aqueous layer was treated with carbon, neutral alumina at 10-15° C. and filtered. The filtrate was mixed with methanol at −20° C. followed by the addition of ethyl acetate and stirred. The solid obtained was filtered, washed with a mixture of cyclohexane:ethanol (200 ml) and dried under vacuum. Yield: 41 g; Purity by HPLC: 99.13%; Palladium content: 1.71 ppm by ICP MS

### Example-V

### Preparation of Ertapenem Monosodium of Formula (I)

To the Step-I organic layer as provided in Example-I, 3-(N-morpholino)propanesulfonic acid solution was added and subjected to hydrogenation using palladium on carbon at 8-10° C. with 9-10 kg hydrogen pressure. The reaction mass, after completion of reaction, was filtered and the filtrate was treated with thiourea and 2-methyltetrahydrofuran and the layers separated. The aqueous layer was treated with carbon, neutral alumina at 10-15° C. and filtered. The filtrate was mixed with methanol at −20° C. and the pH was adjusted to 5-6 using aqueous acetic acid. To the mass, a mixture of ethyl acetate containing 10% methyl acetate was added and stirred. The solid obtained was filtered, washed with cyclohexane:ethanol and dried under vacuum. Yield: 40.5 g; Purity by HPLC: 98.77%; Palladium content: 1.43 ppm by ICP MS

### Example-VI

### Preparation of Meropenem of Formula (VI)

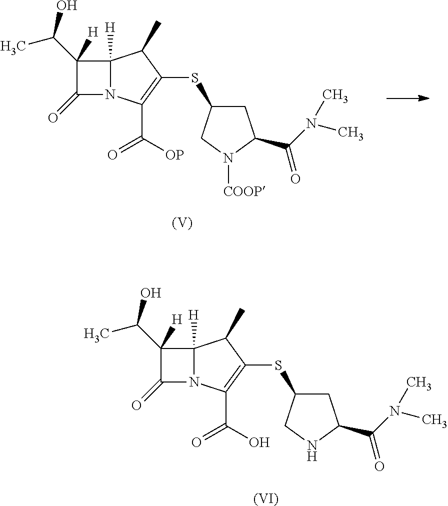

The diprotected Meropenem of formula (V) (where P and P′ were p-nitrobenzyl) was dissolved in tetrahydrofuran and 3-(N-morpholino)propanesulfonic acid buffer and hydrogenated using palladium on carbon at 9-10 kg hydrogen pressure. The mass was filtered and the filtrate was washed with ethyl acetate. The aqueous layer was treated with thiourea and 2-methyltetrahydrofuran. The aqueous layer was separated, treated with carbon and degassed. The carbon was filtered off and acetone was added to the filtrate to crystallize Meropenem trihydrate of formula (VI). The product was filtered and washed with aq. acetone and dried under vacuum to get Meropenem trihydrate. Purity: 99.8%; Pd content: 0.08 ppm

### Reference Example-I

### Preparation of Ertapenem Monosodium of Formula (I)

To Step-I organic layer as provided in Example-I, 3-(N-morpholino)propanesulfonic acid solution was added and hydrogenated at 9-10 kg pressure using palladium on carbon at 8-10° C. The reaction mass, after completion of reaction, was filtered. The filtrate was treated with thiourea and tetrahydrofuran and the layers separated. The aqueous layer was treated with carbon and neutral alumina at 10-15° C. and filtered. The filtrate was mixed with methanol at −20° C. and the pH was adjusted to 5.5-5.7 using aqueous acetic acid. To the mass ethyl acetate was added and stirred. The solid obtained was filtered, washed with ethanol (5*100 ml) and dried under vacuum. Yield: 31 g; Purity by HPLC: 96.76%

### Reference Example-II

### Preparation of Ertapenem Monosodium of Formula (I)

To the Step-I reaction mass as provided in Example-I, 3-(N-morpholino)propanesulfonic acid solution was added and hydrogenated at 9-10 kg pressure using palladium on carbon at 8-10° C. The reaction mass, after completion of reaction was filtered and the layers separated. The aqueous layer was treated with carbon and neutral alumina at 10-15° C. and filtered. The filtrate was mixed with methanol at −20° C. and the pH was adjusted to 5.5-5.7 using aqueous acetic acid. To the mass, ethyl acetate was added and stirred. The solid obtained was filtered, washed with a mixture of cyclohexane:ethanol and dried under vacuum. Yield: 43 g; Purity by HPLC: 98.6%; Palladium content: 35.8 ppm by ICP MS.

### Reference Example-III

### Preparation of Ertapenem Monosodium of Formula (I)

To the Step-I reaction mass as provided in Example-I, 3-(N-morpholino)propanesulfonic acid solution was added and hydrogenated at 9-10 kg pressure using palladium on carbon at 8-10° C. The reaction mass, after completion of reaction, was filtered and the layers separated. The aqueous layer was treated with carbon, neutral alumina at 10-15° C. and filtered. The filtrate was mixed with 1-propanol at −5° C. and the pH was adjusted to 5.5-5.7 using aqueous acetic acid. To the mass methanol and 1-propanol were added and stirred. The solid obtained was filtered, washed with ethanol and dried under nitrogen atmosphere in vacuum. Yield: 25 g; Purity by HPLC: 97%: palladium content: 38.2 ppm

The following tables illustrate the advantages of the present invention over prior art process:

TABLE I
Comparison of present process with prior art process

| | (Reference | Present process |
|---|---|---|
| Parameter | example-III*) | (Example-I) |
| Crystallization solvent | Water:Methanol:1- | Water:Methanol:Ethyl |
| system | Propanol | acetate |
| Washing solvent | Ethanol | Cyclohexane-Ethanol |
| Description | Yellow color powder | Off white color powder |
| Yield | 0.25 w/w | 0.46 w/w |
| Purity (by HPLC) | 97% | 98.93% |
| Palladium content | 38.2 ppm | 1.8 ppm |
| (by ICP MS) | | |
| Filtration time | 180 min | 20 min |
| Washing & Drying | 360 min | 120 min |
| time | | |

*The crystallization and washing method disclosed in U.S. Pat. No. 7,022,841 was followed.

The above table indicates that the use of ethyl acetate as crystallization solvent results with improved yield and high purity with less filtration and drying time thereby increasing the productivity significantly on manufacturing scale. Further the use of thiourea or thiosemicarbazide as reagents in the present process results in the pharmaceutically acceptable level of palladium content.

TABLE II
Comparison of solvents for washing Ertapenem monosodium

| | Cyclohexane:Ethanol | n-heptane:Ethanol | Toluene:Ethanol | Cyclo | |
|---|---|---|---|---|---|
| Parameter | (50:50) | (50:50) | (50:50) | hexane | Ethanol |
| Yield | 0.46 w/w | 0.43 w/w | 0.42 w/w | 0.44 w/w | 0.31 w/w |
| Purity | 98.93% | 99.09% | 99.03% | 98.84% | 96.76% |

The above table indicates that the use of hydrocarbon solvents containing 0-75% of alcoholic solvent helps in washing to remove the residual solvent content in shorter duration and with single run wash. On the other hands the use of ethanol alone results in Ertapenem monosodium having less yield and purity requiring repetitive washing.

TABLE III
Effect of different reagent in reduction of palladium content

| | | | Palladium |
|---|---|---|---|
| | Experiment | Reagent/solvent used | content |
| 1 | Example-I | Thiourea/tetrahydrofuran | 1.8 ppm |
| 2 | Example-II | Thiourea/2-methyl | 0.93 ppm |
| | | tetrahydrofuran | |
| 3 | Example-IV | Thiosemicarbazide/ | 1.71 ppm |
| | | tetrahydrofuran | |
| 4 | Reference example-II | No reagent | 35.8 ppm |
| 5 | Reference example- | No reagent | 38.2 ppm |
| | III (prior art process) | | |

Reagent: thiourea, thiosemicarbazide or its N-substituted derivatives

Advantages of the Process of the Present Invention:
The use of ester of an organic acid for the crystallization of Ertapenem sodium results in fast filtration and reduced cycle time, thereby increasing the productivity.
Washing of Ertapenem sodium with hydrocarbon solvent optionally containing alcohol results in improved physical nature of Ertapenem sodium resulting in reduced washing and drying time thereby avoid the degradation of Ertapenem and providing Ertapenem sodium with purity greater than 98.5% by HPLC.
Use of thiourea, thiosemicarbazide or their N-substituted derivatives in the process results in Ertapenem sodium having pharmaceutically acceptable level of palladium content.

1. An improved process for the preparation of Ertapenem monosodium of formula (I) having the purity greater than 98.5% 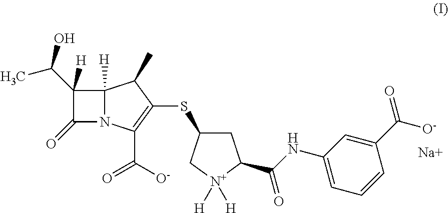 [SMILES: C[C@@H](O)[C@H]1C(=O)N2C(C(=O)[O-])=C(S[C@@H]3C[NH2+][C@H](C(=O)Nc4cccc(C(=O)[O-])c4)C3)[C@H](C)[C@H]12 ; [Na+]]
the said process comprises the steps of:
(a) obtaining an aqueous solution containing Ertapenem monosodium of formula (I);
(b) optionally contacting the solution with thiourea or thiosemicarbazide or their N-substituted derivatives in the presence of organic solvent;
(c) adding step (a) or step (b) mass to alcoholic solvent or vice-versa;
(d) mixing ester of an organic acid with the step (c) mass;
(e) filtering the solid; and optionally washing the solid with hydrocarbon solvent containing 0-75% alcohol; wherein the improvement is characterized by one or more of the following:
1. use of thiourea or thiosemicarbazide or its N-substituted derivatives in step (b)
2. use of ester of an organic acid in step (d)
3. use of hydrocarbon solvent containing 0-75% alcoholic solvent as washing solvent in step (e).

2. The process as claimed in claim 1, wherein the alcoholic solvents used in step (c) is selected from a group consisting of methanol, ethanol or isopropyl alcohol or mixtures thereof, preferably methanol.

3. The process as claimed in claim 1, wherein the ester of an organic acid used in step (d) is selected from ethyl acetate, methyl acetate, n-propyl acetate, isopropyl acetate, n-butyl acetate or mixtures thereof, preferably ethyl acetate.

4. The process as claimed in claim 1, wherein the hydrocarbon solvent used in step (e) is selected from cyclohexane, n-hexane, n-heptane, cyclopentane, decalin, or toluene or mixtures thereof; and alcohol is selected from methanol, methoxy ethanol, isopropanol, ethanol or mixtures thereof; preferably a mixture of cyclohexane and ethanol.

5. A process for reducing the palladium content in penem/penam antibiotic comprising treating the aqueous solution of penem/penam antibiotic with thiourea or thiosemicarbazide or their N-substituted derivatives in the presence of organic solvent followed by isolating the penem/penam antibiotic from aqueous layer.

6. The process as claimed in claim 5, wherein the penem/penam antibiotic is selected from Ertapenem or it monosodium salt, Meropenem or its hydrate, Imipenem or its hydrate, Biapenem or its hydrate and Doripenem or its hydrate, Tebipenem, Faropenem, Tazobactam.

7. The process as claimed in claim 1, wherein the organic solvent used in the presence of thiourea or thiosemicarbazide or their N-substituted derivatives is selected from tetrahydrofuran, 2-methyltetrahydrofuran, ethyl acetate, acetonitrile or mixtures thereof.

8. Ertapenem monosodium characterized by X-ray powder diffraction pattern as given in FIG. 1.

9. An improved process for the preparation of Ertapenem monosodium having pharmaceutically acceptable level of residual organic solvent comprises washing the Ertapenem monosodium containing residual solvents selected from the group consisting of with hydrocarbon solvent containing 0 to 75% of alcoholic solvent.

10. The process as claimed in claim 9, wherein the washing step reduces the residual solvent content to pharmaceutically acceptable level.

11. The process as claimed in claim 9, wherein the residual solvent reduced is selected from solvent consisting of ethyl acetate, methanol, tetrahydrofuran, 2-methyltetrahydrofuran, dimethylformamide, dimethylacetamide, 1-propanol, isopropanol, dichloromethane, methyl acetate, propyl acetate, butyl acetate, acetonitrile, toluene, 1,2-dibromoethane or mixture thereof

12. A process as claimed in claim 9, wherein the hydrocarbon solvent is selected from cyclohexane, n-hexane, n-heptane, cyclopentane, decalin, toluene or mixtures thereof; and alcohol is selected from methanol, methoxy ethanol, isopropanol, ethanol or mixtures thereof; preferably a mixture of cyclohexane and ethanol.

13. The process according to claim 1, substantially as hereinbefore described with reference to examples 1 to 4.

14. The process as claimed in claim 5, wherein the organic solvent used in the presence of thiourea or thiosemicarbazide or their N-substituted derivatives is selected from tetrahydrofuran, 2-methyltetrahydrofuran, ethyl acetate, acetonitrile or mixtures thereof.
